(19) United States
(12) Patent Application Publication
(10) Pub. No.: US 2012/0283202 A1
(43) Pub. Date: Nov. 8, 2012

(54) ANTI-WART PHARMACEUTICAL COMPOSITION AND METHOD FOR TREATING WART

(75) Inventors: Kou-Wha Kuo, Kaohsiung City (TW); Hamm-Ming Sheu, Kaohsiung City (TW)

(73) Assignee: G & E HERBAL BIOTECHNOLOGY CO., LTD., Tainan City (TW)

(21) Appl. No.: 13/368,835

(22) Filed: Feb. 8, 2012

(30) Foreign Application Priority Data
May 4, 2011 (TW) 100115591

(51) Int. Cl. A61K 31/706; A61P 17/12

(52) U.S. Cl. 514/26

(57) ABSTRACT

Disclosed herein is an anti-wart pharmaceutical composition including a water-soluble extract from a plant of Solanum genus, the water-soluble extract comprising solamargine and solasonine. Also disclosed is a method for treating wart including applying to a subject in need of such treatment the water-soluble extract.

### CROSS-REFERENCE TO RELATED APPLICATION

This application claims priority of Taiwanese application no. 100115591, filed on May 4, 2011.

### BACKGROUND OF THE INVENTION

1. Field of the Invention

This invention relates to an anti-wart pharmaceutical composition containing a water-soluble extract from a plant of Solanum genus and a method for treating wart using the water-soluble extract.

2. Description of the Related Art

Wart is a kind of dermatosis that occurs on the skin or mucous of human or animal subjects, and is a benign epidermal hyperplasia caused by infection of Human papilloma virus (HPVs). In general, based on the site where the wart forms, wart can be divided into cutaneous wart and mucosal wart. The epidermal cells of skin or mucosa affected by HPVs are subjected to benign proliferation and hyperkeratosis, thereby resulting in formation of papules or nodules with roughened surfaces on the skin or mucosa.

In clinical diagnosis, wart is divided into four types according to symptoms thereof: (1) common wart (Verruca vulgaris), scaly and roughened papule(s) or nodule(s) which usually occur on hands or fingers; (2) flat wart (Verruca plana juvenilis), slightly raised and flat-topped papule(s) which usually occurs on faces, hands, and lower legs; (3) plantar wart (Verruca plantaris), a thick and hyperkeratotic papule which usually occurs at pressure points of soles and toes and causes painful feeling for a patient when the papule is pressed; and (4) genital wart (Condyloma acuminatum), a cauliflower-like lump formed on skin or mucosa of genitalia and anus, which is a highly infectious and sexually transmitted disease (STD).

Nowadays, warts are treated using the following remedies based on the type, size, number, and lesion site thereof:

(1) Topical therapy, e.g., podophyllin (Trade name: Wartec®) and imiquimod (trade name: Aldara®) for treating genital wart, 5-fluorouracil for treating genital wart and cutaneous wart, and bleomycin (Trade name: Bleocin®) for treating plantar wart and warts formed on palm;

(2) Laser treatment, e.g., pulsed dye laser and carbon dioxide laser, especially for treating plantar wart and warts formed on palm; and

(3) Cryotherapy using liquid nitrogen and suitable for treating most warts, less effective for warts formed on palm, sole, and toe.

Each of the aforesaid remedies is suitable only for a specific type of wart, rather than all types of warts. Moreover, in clinical diagnosis, there are inferior effect problem and side effect drawback for the aforesaid remedies. For example, topical therapy used for a long period of time will cause recurrence of warts and treating effect thereof is unsatisfactory. Laser treatment and cryotherapy will cause severe side effects, such as pain, ulceration, and scarring. Some patients, especially children, are likely to refuse treatment due to the unendurable severe side effects.

To overcome the aforesaid drawbacks, researches have focused on finding active components from traditional Chinese medicines or herbs to develop a drug that is effective for treating warts and that has no undesired side effects.

It is known that Solanum incanum L. (also known as Solanum incanum Ruiz. & Pay., Solanum undatum, Solanum coagulans Forsskal in Latin, and bitter apple in English) contains steroidal glycoalkaloid which exhibits anti-cancer activity. In addition, many plants of the Solanum genus are reported to contain steroidal glycoalkaloid, including, for example, Solanum indicum, Solanum nigrum, also known as Long Kui in Chinese and black nightshade in English, Solanum capsicastrum (known as false Jerusalem cherry in English), Solanum xanthocarpum, Solanum melongena, Solanum coagulans, Solanum tuberosum, Solanum sodomeum, Solanum turburosum, Solanum aculeastrum, Solanum lycocarpum, Solanum khasianum, Solanum suaveolens, Solanum uporo, Solanum abutiloides, Solanum coccineum, Solanum unguiculatum, Solanum robustum, Solanum anguivi, Solanum platanifolium, Solanum mammosum, etc. The common steroidal glycoalkaloids contained in plants of Solanum genus include solamargine and solasonine.

U.S. Pat. No. 7,078,063 B2 issued to the inventors of this invention discloses a water soluble extract from a plant of Solanum genus, especially Solanum incanum L., and a process for preparing the same. The water soluble extract consists essentially of at least 60 wt % of solamargine and solasonine. This patent is hereby incorporated by reference in its entirety.

In the aforesaid US patent, the inventors found that the water soluble extract exhibits inhibition of growth of tumor/cancer cells, in particular liver tumor cells, lung cancer cells, and breast cancer cells. In this invention, the inventors unexpectedly found that the water soluble extract is effective in treating warts.

### SUMMARY OF THE INVENTION

Therefore, according to a first aspect, this invention provides an anti-wart pharmaceutical composition which includes a water-soluble extract from a plant of Solanum genus, the water-soluble extract comprising solamargine and solasonine.

In a second aspect, this invention provides a method for treating wart, which comprises applying to a subject in need of such treatment a water-soluble extract from a plant of Solanum genus, the water-soluble extract comprising solamargine and solasonine.

### BRIEF DESCRIPTION OF THE DRAWINGS

The above and other objects, features and advantages of this invention will become apparent with reference to the following detailed description and the preferred embodiments taken in conjunction with the accompanying drawings, in which:

FIG. 1 shows photos illustrating that lesions (indicated by arrows) of test subject C are gradually ameliorated by treating the same with a Solanum incanum L. gel;

FIG. 2 shows photos illustrating that lesions (indicated by arrows) of test subject D are gradually ameliorated by treating the same with a Solanum incanum L. gel;

FIG. 3 shows photos illustrating that lesions (indicated by arrows) of test subject E are gradually ameliorated by treating the same with a Solanum incanum L. gel; and

FIG. 4 shows photos illustrating that lesions (indicated by arrows) of test subject F are gradually ameliorated by treating the same with a Solanum incanum L. gel.

### DETAILED DESCRIPTION OF THE PREFERRED EMBODIMENTS

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the words “comprises,” “contain” and variants thereof have a corresponding meaning.

Unless otherwise defined, all technical and scientific terms used herein have the meaning commonly understood by a person skilled in the art to which this invention belongs. One skilled in the art will recognize many methods and materials similar or equivalent to those described herein, which could be used in the practice of this invention. Indeed, this invention is in no way limited to the methods and materials described. For clarity, the following definitions are used herein.

The present invention provides an anti-wart pharmaceutical composition, which includes a water-soluble extract from a plant of Solanum genus. The water-soluble extract comprises solamargine and solasonine. Preferably, the water-soluble extract comprises at least 60 wt % of solamargine and solasonine, more preferably, 60 wt %-90 wt % of solamargine and solasonine.

The process for preparing the water-soluble extract has been disclosed in U.S. Pat. No. 7,078,063 B2 and includes the steps of:
(a) subjecting a plant material of a plant of Solanum genus to an extraction treatment using an acidic aqueous solution with a pH value of 3˜5, such that an aqueous solution is obtained;
(b) adjusting the pH value of the aqueous solution obtained in step (a) to pH 8˜10 with a base, such that a precipitate is formed;
(c) washing the precipitate formed in step (b) with water, followed by drying, such that a dried product is obtained;
(d) admixing the dried product obtained in step (c) with chloroform, followed by addition of a suitable amount of a 100% alcohol, such that a chloroform-alcohol mixture is formed;
(e) mixing the chloroform-alcohol mixture formed in step (d) with a water/alcohol solution having a predetermined water:alcohol ratio, such that mixture containing a chloroform-based layer and a non-chloroform-based layer is obtained;
(f) removing the chloroform-based layer from the mixture obtained in step (e), followed by addition of a suitable amount of water; and
(g) obtaining a supernatant from the resultant mixture of step (f), followed by drying the supernatant, wherein the resultant dried product is able to be directly dissolved in water to form a yellowish clear and transparent aqueous solution.

Preferably, in step (a), the plant material of the plant of Solanum genus has been chopped in a preliminary treatment.

Preferably, in step (a), the plant material is at least one of the fruit, root, stem, and leaf of the plant of Solanum genus. In a preferred embodiment of this invention, the plant material used in step (a) is the fruit of the plant of Solanum genus.

The inventors found that certain factors might affect the content and proportion of solasonine and solamargine in the water-soluble extract obtained using the aforesaid process. These factors include the species of the plant of Solanum genus and the part/parts of the plant used in the extracting process, as well as the types of alcohol and base used. Therefore, a skilled artisan can prepare a desired water-soluble extract by selecting a suitable species of the plant of Solanum genus and using a suitable part or parts of the plant, in conjunction with appropriate operating conditions.

Preferably, the water-soluble extract is obtained from a plant of Solanum genus selected from the group consisting of Solanum incanum L., Solanum indicum, Solanum nigrum, Solanum capsicastrum, Solanum xanthocarpum, Solanum melongena, Solanum coagulans, Solanum tunigrum, Solanum sodomeum, Solanum turburosum, Solanum aculeastrum, Solanum lycocarpum, Solanum khasianum, Solanum suaveolens, Solanum uporo, Solanum abutiloides, Solanum coccineum, Solanum unguiculatum, Solanum robustum, Solanum anguivi, Solanum platanifolium, and Solanum mammosum. In a preferred embodiment of this invention, the water-soluble extract is obtained from Solanum incanum L.

In preliminary human tests done by the inventors, the results show that the water-soluble extract is effective in treating or alleviating warts including cutaneous warts and mucosal warts without undesired side effects, e.g., ulceration or erythema. Therefore, the present invention provides a method for treating wart, which comprises applying to a subject in need of such treatment the water-soluble extract.

The wart that can be treated by the anti-wart pharmaceutical composition of this invention includes common wart, flat wart, plantar wart, and genital wart.

The anti-wart pharmaceutical composition of this invention can be administered via e.g., parenteral, transmucosal, transdermal, or topical route.

The parenteral route includes subcutaneous injection, intraepidermal injection, intradermal injection, and intralesional injection.

For the parenteral route, the anti-wart pharmaceutical composition of this invention can be formulated into an injection product using techniques known to a skilled artisan, e.g., sterile aqueous solution or dispersion.

In this invention, the injection product can be prepared by mixing the water-soluble extract with a pharmaceutically acceptable carrier that is widely employed in drug-manufacturing technology.

The pharmaceutically acceptable carrier comprises one or more reagents, including, for example, water, saline, buffer solution (Phosphate Buffered Saline (PBS), Ringer's solution or Hank's solution), emulsifier, suspending agent, decomposer, pH adjusting agents, stabilizing agent, chelating agent, preservative, diluents, absorption delaying agent, liposome, etc. The choice and amount of these pharmaceutically acceptable carriers are within the expertise of those skilled in the art.

Moreover, the anti-wart pharmaceutical composition of this invention can be administered by topical routes, e.g., dermal, buccal, genital, vaginal, anal, and rectal routes. In an embodiment of this invention, the anti-wart pharmaceutical composition of this invention is administered by dermal route. In another embodiment of this invention, the anti-wart pharmaceutical composition of this invention is administered by genital route.

The pharmaceutical composition according to this invention can be formulated into a suitable dosage form for topical administration using technology well known to those skilled in the art, which includes, but is not limited to, external preparations, effervescent tablets, suppositories, and the like.

In a preferred embodiment of this invention, the anti-wart pharmaceutical composition is formulated into an external preparation by admixing the water-soluble extract according to this invention with a base that is well known and commonly used in the art.

In this invention, the external preparation includes, but is not limited to, emulsion, gel, ointment, cream, patch, liniment, powder, aerosol, spray, lotion, serum, paste, foam, drop, suspension, and tincture. In an embodiment of this invention, the anti-wart pharmaceutical composition is in the form of gel.

For example, the base suitable for producing the external preparation according to this invention may include one or more of the following additives: water, alcohols, glycol, hydrocarbons (e.g., petroleum jelly and white petrolatum), wax (e.g., paraffin and yellow wax), preserving agents, antioxidant, surfactants, absorption enhancers, stabilizing agents, gelling agents (e.g., Carbopo®974P, microcrystalline cellulose, and carboxymethyl cellulose), active agents, humectants, odor absorbers, fragrances, pH adjusting agents, chelating agent, emulsifier, occlusive agents, emollients, thickeners, solubilizing agents, penetration enhancers, anti-irritants, colorants, and propellants. The choice and amount of these additives are within the expertise of those skilled in the art.

This invention also provides a method for treating wart, which comprises applying to a subject in need of such treatment the water-soluble extract or the anti-wart pharmaceutical composition.

The dosage and the frequency of administration of the anti-wart pharmaceutical composition according to this invention may vary depending on the following factors: the severity of the disease to be treated, the route of administration, and the weight, age, physical condition and response of the subject to be treated. For instance, the daily dosage of the pharmaceutical composition according to this invention may be 10 to 20 mg/cm² of the lesion area and one to six times per day.

This invention will be further described by way of the following examples. However, it should be understood that the following examples are solely intended for the purpose of illustration and should not be construed as limiting the invention in practice.

### EXAMPLES

### Example 1

### Preparation of the Water-Soluble Extract

The water-soluble extract was prepared based on the procedure as disclosed in Example 1 of U.S. Pat. No. 7,078,063 B2. Specifically, 500 g of ripe fruit of Solanum incanum L. was ground subsequent to addition of 1000 ml pure water. To the resultant aqueous mixture, 99.5% of acetic acid was added dropwise to adjust the pH value to 4.0, followed by shaking at room temperature for 12 hrs. A supernatant was obtained by centrifuging the aqueous mixture at 4500 rpm for 12 hours, and 33% NH₄OH basic solution was added thereto dropwise to adjust the pH value of the supernatant to 9.0, and a precipitate was formed. The precipitate was obtained by conducting centrifugation at 4,500 rpm for 10 minutes (Beckman Coulter, Avanti J-25, JA-14 Rotor), and the residual basic solution present therein was removed by washing the precipitate with water, followed by centrifugation at 4,500 rpm. The precipitate thus obtained was suspended in distilled water and subjected to lyophilization (Virtis, Freezemobile 12ES) to obtain 5 g of dried powder.

2 g of the dried powder was dissolved in 50 ml chloroform in reagent grade, followed by addition of 40 ml of 100% methanol and shaking to form a uniform suspension. A supernatant was obtained by centrifugation at 4,500 rpm. 70 ml of methanol:water solution (1:1) was added to the supernatant and mixed well. The mixture obtained was centrifuged at 12,000 rpm for 10 min. The resultant supernatant was taken out, and 120 ml distilled water was added thereto and shaken well. The supernatant was further centrifuged at 12,000 rpm for 10 min so as to remove the precipitate. The resultant supernatant was subjected to concentration under reduced pressure at 55° C. to remove methanol, followed by lyophilization to obtain dried powder of the water-soluble extract.

### Example 2

### Therapeutic Effect and Safety of the Water-Soluble Extract in Treating Human Cutaneous Wart and Mucosal Wart

In order to examine the therapeutic effect and safety of the water-soluble extract as prepared in the above Example 1 in treating human cutaneous wart and mucosal wart, the following tests were conducted.

### A. Preparation of Gel Containing the Water-Soluble Extract

4 g of Carbopol®974P, which was used as a gelling agent and is commercially available from Lubrizol Advanced Materials, Inc., KY 40258, USA, was dissolved in 50 g pure water, followed by sequential addition of 30 g of propylene glycol and 7 g of the dried powder of the water-soluble extract obtained in Example 1 and mixing well. The mixture was heated in a heating vessel at a temperature of 50° C. to 60° C. for 20 minutes, followed by cooling at room temperature. The cooled mixture was added with triethanolamine to adjust pH to 7.0±0.5. Subsequently, water was added until the total weight of the mixture reached 100 g, thereby obtaining a gel containing 7% (w/w) of the water-soluble extract (hereinafter referred to as Solanum incanum L. gel).

### B. Test Subjects and Clinical Information Thereof.

Six test subjects (test subjects A to F) participating in the following tests were enrolled from the outpatient department of National Cheng Kung University Hospital, Taiwan. The test subjects A and B suffered from mucosal wart, and the test subjects C to F suffered from cutaneous wart. The clinical information of the six test subjects, including gender, age, wart type, and lesion location, are outlined in Table 1.

TABLE 1

| Test | Age | | | |
|---|---|---|---|---|
| subject | (y/o) | Gender | Wart type | Lesion location |
| A | 32 | Female | Mucosal wart | External genital |
| B | 21 | Female | Mucosal wart | External genital |
| C | 28 | Female | Cutaneous wart | Finger tip |
| D | 21 | Male | Cutaneous wart | Finger tip, finger |
| E | 26 | Male | Cutaneous wart | Finger |
| F | 33 | Male | Cutaneous wart | Finger tip, palm |

### C. Pre-Clinical Test

Prior to the start of the test (i.e., on Week 0), selected lesions on each of the enrolled test subjects were photographed. The Solanum incanum L. gel prepared in section A of Example 2 was applied to the selected lesions and normal skin areas that surround the selected lesions. Each of the normal skin areas had inner and outer peripheries and the distance between the inner and outer peripheries of each of the normal skin areas was not greater than 0.5 cm. The treated dosage was 10˜20 mg/cm², and twice a day. The selected lesions of each of the test subjects were photographed at predetermined evaluation times (i.e., at the ends of weeks 1, 2, 3, 4, 7, 8, 13, 20 and 29, see Table 2), and evaluations for therapeutic effect and safety of the Solanum incanum L. gel were conducted according to the procedures set forth in sections D and E below. The test was finished when the lesions of each of the test subjects were completely cured and the outer appearance of the lesion skin was recovered to a normal state.

TABLE 2

| Test | Evaluation time (week) | | | | | | | | | |
|---|---|---|---|---|---|---|---|---|---|---|
| subject | 0^{a} | 1^{b} | 2 | 3 | 4 | 7 | 8 | 13 | 20 | 29 |
| A | +^{c} | + | −^{d} | − | − | − | − | − | − | − |
| B | + | + | − | − | − | − | − | − | − | − |
| C | + | − | − | − | + | − | + | − | − | − |
| D | + | − | + | + | − | − | + | − | − | − |
| E | + | − | + | − | − | + | − | − | − | − |
| F | + | − | − | − | − | + | − | ⊚^{e} | ^{f} | + |

^{a}“0” indicates week 0, i.e., prior to application of the Solanum incanum L. gel
^{b}“1” means week 1 after application of the Solanum incanum L. gel
^{c}“+” means evaluation was conducted
^{d}“−”means no evaluation was conducted
^{e}“⊚” means evaluation was conducted only on the lesions at finger tip
^{f}“”means evaluation was conducted only on the lesion on palm

### D. Evaluation of Therapeutic Effect

The photos of the selected lesions of the test subjects as obtained at Week 0 (i.e., prior to the application of the Solanum incanum L. gel) and as obtained at the evaluation times were compared by a dermatologist to determine the improvement of lesions treated with the Solanum incanum L. gel.

### E. Evaluation of Safety

Evaluation of safety was conducted by a researcher at the predetermined evaluation times. The test subjects were interrogated by the researcher in respect to the appearance of any adverse skin response (including ulceration, irritation and erythema).

### Results:

A. Evaluation of the Therapeutic Effects of the Solanum incanum L. Gel

Compared with the photos of the selected lesions of each of the test subjects A and B as obtained at week 0 and other predetermined evaluation times, the selected lesions of each of the test subjects A and B treated with the Solanum incanum L. gel were significantly improved after the end of week 1 (data not shown).

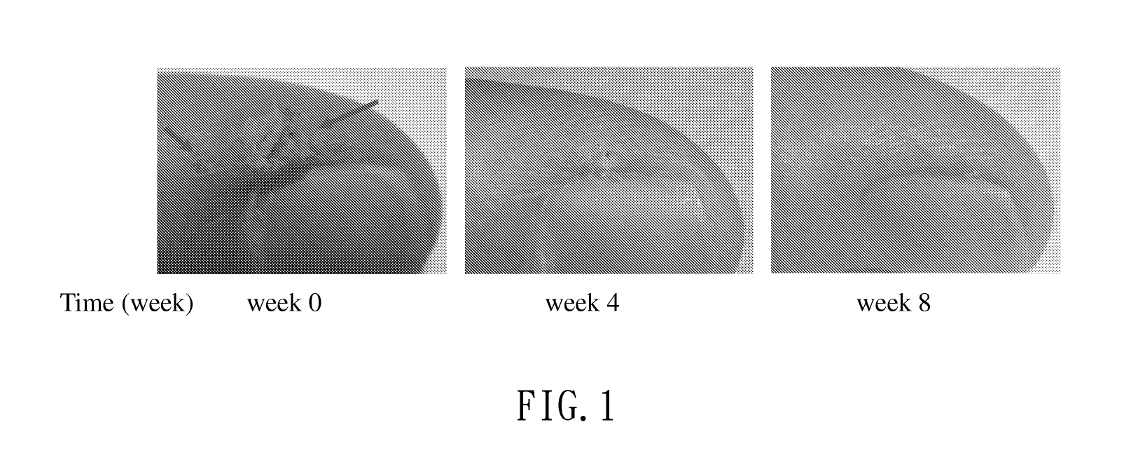

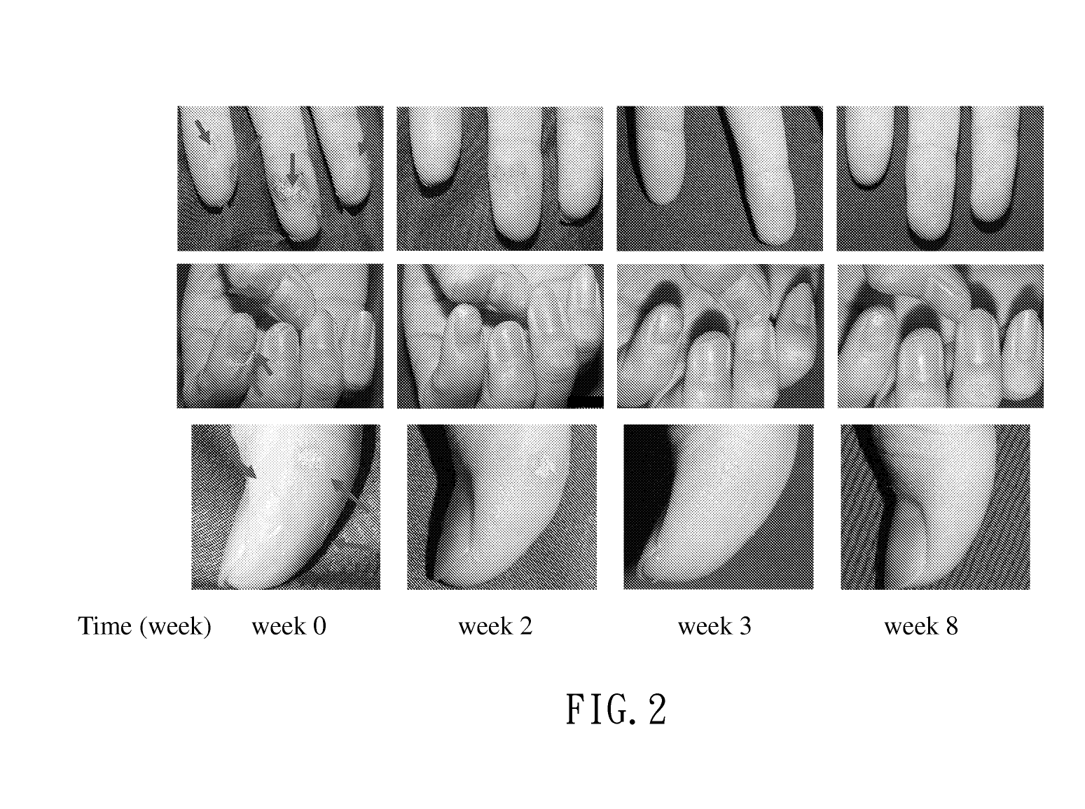

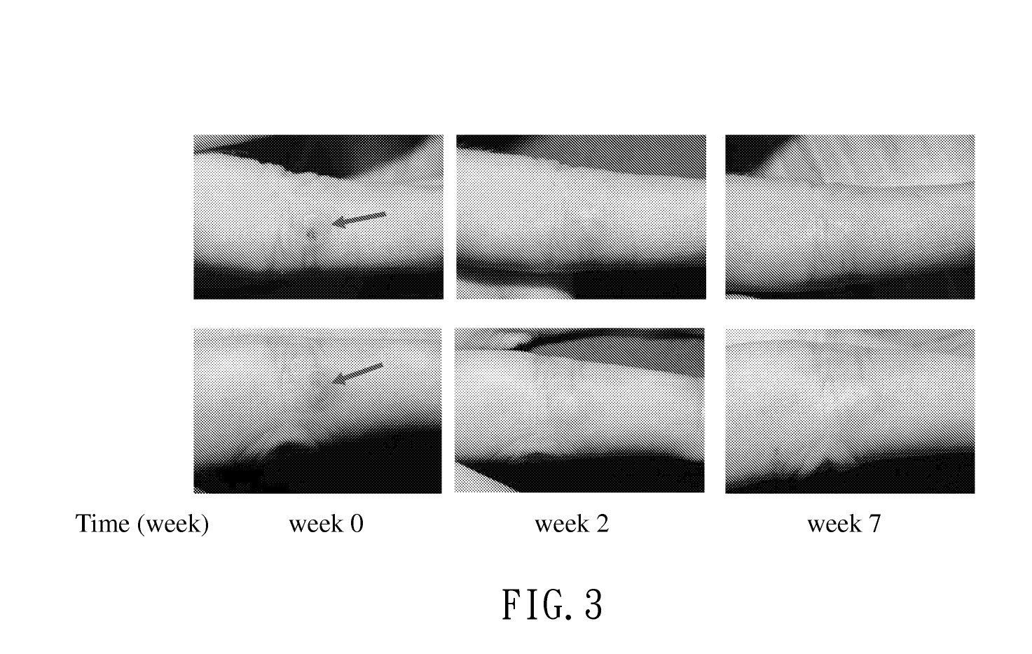

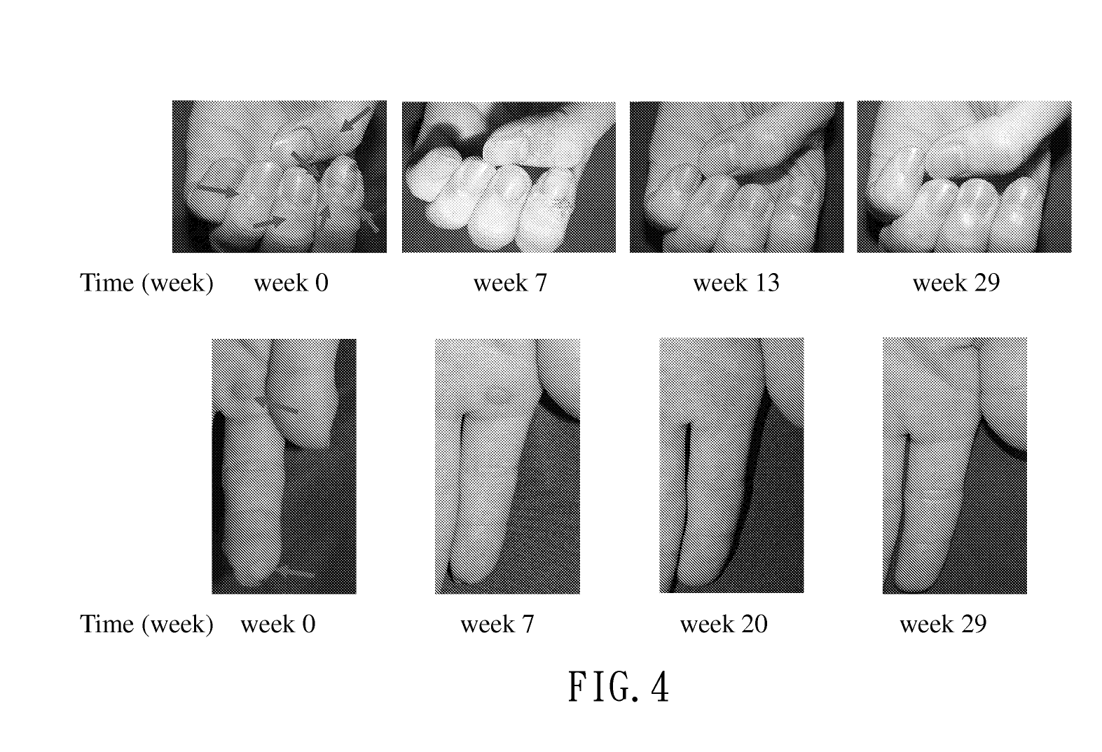

FIGS. 1 to 4 respectively show photos of the selected lesions of the test subjects C to F as obtained at different evaluation times. The photos demonstrate that the lesions of each of the test subjects C to F treated with the Solanum incanum L. gel greatly improved with time. After the test was finished, the test subjects C to F were further subjected to the follow-up study for six months to two years to determine whether the lesion area that had already been cured by the Solanum incanum L. gel was recurrent. Based on the results of the follow-up study, no warts were found to have recurred for all of the test subjects (data not shown).

The aforesaid results show that the Solanum incanum L. gel of this invention could be effectively used to treat cutaneous wart and mucosal wart.

### B. Evaluation of the Safety of the Gel Containing the Water-Soluble Extract

In the safety test, only a few of the test subjects experienced temporary and mild adverse skin responses, including irritation or erythema, which, however, did not halt them from completing the test (data not shown).

In view of the aforesaid, the inventors contemplate that the water-soluble extract from a plant of Solanum genus according to this invention can be developed into an anti-wart drug for long-term use in treating warts without adverse side effects.

All patents and literature references cited in the present specification as well as the references described therein, are hereby incorporated by reference in their entirety. In case of conflict, the present description, including definitions, will prevail.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. An anti-wart pharmaceutical composition, comprising a water-soluble extract from a plant of Solanum genus, said water-soluble extract comprising solamargine and solasonine.

2. The pharmaceutical composition of claim 1, wherein said water-soluble extract comprises at least 60 wt % of solamargine and solasonine.

3. The pharmaceutical composition of claim 1, wherein said water-soluble extract comprises 60 wt %-90 wt % of solamargine and solasonine.

4. The pharmaceutical composition of claim 1, which is used for treating common wart, flat wart, plantar wart, or genital wart.

5. The pharmaceutical composition of claim 1, which is applied in a topical dosage form.

6. The pharmaceutical composition of claim 5, further comprising a base selected from the group consisting of water, alcohols, glycol, hydrocarbons, wax, preserving agents, antioxidant, surfactants, absorption enhancers, stabilizing agents, gelling agents, active agents, humectants, odor absorbers, fragrances, pH adjusting agents, chelating agent, emulsifier, occlusive agents, emollients, thickeners, solubilizing agents, penetration enhancers, anti-irritants, colorants, propellants, and combinations thereof.

7. The pharmaceutical composition of claim 6, wherein the base includes a gelling agent and glycol.

8. A method for treating wart, comprising applying to a subject in need of such treatment a water-soluble extract from a plant of Solanum genus, the water-soluble extract comprising solamargine and solasonine.

9. The method of claim 8, wherein the water-soluble extract comprises at least 60 wt % of solamargine and solasonine.

10. The method of claim 8, wherein the water-soluble extract comprises 60 wt %-90 wt % of solamargine and solasonine.

11. The method of claim 8, wherein the wart is common wart, flat wart, plantar wart, or genital wart.

12. The method of claim 8, wherein the water-soluble extract is applied in a topical dosage form.
